(19) United States
(12) Patent Application Publication
(10) Pub. No.: US 2017/0209272 A1
(43) Pub. Date: Jul. 27, 2017

(54) Method Of Detecting Cancer Based On Spermine/Spermidine N'-Acetyltransferase Gene Expression

(71) Applicant: BioMark Technologies Inc., Richmond (CA)

(72) Inventors: Rashid Bux, Vancouver (CA); Brian Cheng, Chesterfield, MO (US); Paramjit S. Tappia, Winnipeg (CA); Bram Ramjiawan, Winnipeg (CA)

(73) Assignee: BioMark Technologies Inc., Richmond, BC (CA)

(21) Appl. No.: 15/317,932

(22) PCT Filed: Jun. 9, 2015
(86) PCT No.: PCT/CA15/50534

§ 371 Date: Dec. 9, 2016

Related U.S. Application Data
(60) Provisional application No. 62/009,864, filed on Jun. 9, 2014.

(51) Int. Cl. A61F 2/28

(52) U.S. Cl. CPC A61F 2/28; A61F 2002/2835; A61F 2230/0006; A61F 2230/0008; A61F 2220/0008; A61F 2210/0076

(57) ABSTRACT

Elevated levels of spermidine/spermine N¹-acetyltransferase (SSAT) gene expression expression were observed in breast, prostate and lung cancer cell lines. Elevated levels of SSAT gene expression were verified in tissues from patients with breast, prostate and lung cancer. Elevated SSAT gene expression is elevated in different human cancers and elevated SSAT gene expression may accordingly serve as a companion diagnostic biomarker for detection and monitoring of cancer progression.

### BACKGROUND OF THE INVENTION

Field of the Invention

The present invention relates to a method of detecting cancer and, in particular, to a method of detecting cancer based on spermidine/spermine N¹-acetyltransferase (SSAT) gene expression.

Description of the Related Art

U.S. Pat. No. 6,811,967 which issued to Sitar et al. on Nov. 4, 2004, and the full disclosure of which is incorporated herein by reference, discloses a method for assaying activity of the enzyme spermidine/spermine N¹-acetyltransferase (SSAT) using SSAT substrates by detecting acetylated forms of the SSAT substrates. The SSAT substrates may include amantadine wherein metabolism of amantadine occurs in part by the action of the inducible enzyme SSAT to produce the acetylated metabolite N-acetylamantadine. Disclosed also is the correlation of SSAT activity to pathological conditions.

### SUMMARY OF THE INVENTION

It is an object of the present invention to provide a method of detecting cancer based on spermidine/spermine N¹-acetyltransferase (SSAT) gene expression.

Elevated levels of SSAT gene expression expression were observed in breast, prostate and lung cancer cell lines. Elevated levels of SSAT gene expression were verified in tissues from patients with breast, prostate and lung cancer. Elevated SSAT gene expression is elevated in different human cancers and elevated SSAT gene expression may accordingly serve as a companion diagnostic biomarker for detection and monitoring of cancer progression.

There is accordingly provided a method of detecting cancer comprising correlating spermidine/spermine N¹-acetyltransferase gene expression to cancer. The method may comprise correlating elevated levels of spermidine/spermine N¹-acetyltransferase gene expression to cancer. The method may comprise correlating spermidine/spermine N¹-acetyltransferase gene expression to breast cancer. The method may comprise correlating spermidine/spermine N¹-acetyltransferase gene expression to lung cancer. The method may comprise correlating spermidine/spermine N¹-acetyltransferase gene expression to prostate cancer.

### BRIEF DESCRIPTIONS OF DRAWINGS

The invention will be more readily understood from the following description of the embodiments thereof given, by way of example only, with reference to the accompanying drawings, in which:

FIG. 1A is a flow chart showing the role of spermidine/spermine N¹-acetyltransferase (SSAT) in polyamine metabolism;

FIG. 1B is a block diagram showing the impact of SSAT inhibition on cancer;

FIG. 2 is a graph showing the relative SSAT expression levels in different human cancer cell lines;

FIG. 3 is a graph showing the relative SSAT expression levels in different patient-derived breast cancer tissue, lung cancer tissue and prostate cancer tissue;

FIG. 4 is a Western blot showing SSAT expression in patient-derived breast cancer tissue;

FIG. 5 is a Western blot showing SSAT expression in patient-derived lung cancer tissue; and

FIG. 6 is a Western blot showing SSAT expression in patient-derived prostate cancer tissue.

### DESCRIPTIONS OF THE PREFERRED EMBODIMENTS

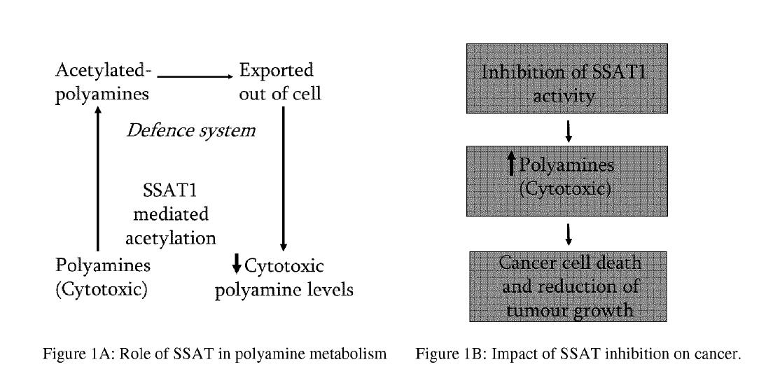

Spermidine/spermine N¹-acetyltransferase (SSAT) is the rate-limiting enzyme in the polyamine metabolic pathway. SSAT plays a regulatory role in spermidine and spermine homeostasis and normally is present in very small amounts in mammalian cells. However, the increased production of polyamines in cancer results in increased levels of polyamines and N¹-acetylspermidine, potentially reflecting increased SSAT activity. The elevation of polyamines triggers the increase in SSAT activity to remove polyamines as part of a cell defense system as shown in FIG. 1A. Since polyamines are cytotoxic, inhibition of SSAT activity would be seen to exert anti-cancer effects by allowing polyamines to cause cancer cell death as shown in FIG. 1B. Based on these observations, SSAT mRNA levels in a selected number of human cancer cell lines and patient-derived breast cancer tissue, prostate cancer tissue, and lung cancer tissue was evaluated.

Total RNA was extracted from the human cancer cell lines and the patient-derived breast cancer tissue, prostate cancer tissue and lung cancer tissue using a Qiagen QIA™ Shredder Kit and RNeasy™ Mini Kit obtained from Life Technologies Inc. of Ontario, Canada. RNA concentration in each extracted sample was confirmed by nanodrop spectrophotometric measurement. RNA integrity was evaluated by measurement of RNA Integrity Number (RIN). SSAT gene expression was determined by qRT-PCR using cDNA probe specific for SSAT and performed using Qiagen QuaniTect™ SYBR Green RT-PCR Kit obtained from Life Technologies Inc. of Ontario, Canada. The mRNA expression levels of the housekeeping genes, glyceraldehyde 3-phosphate dehydrogenase (GAPDH) and hypoxanthine phosphoribosyltransferase 1 (HRPT1), were measured in parallel using the corresponding PCR primers for these genes. The SSAT gene expression levels were normalized with GAPDH or HPRT1 as the internal reference. Normalized SSAT gene expression was further analyzed by the ΔΔct method.

The statistical analysis of the data was performed using SigmaStat (SPSS In.) software. Values were expressed as mean±standard error of the mean. Differences between two groups were evaluated by Student's t-test. A probability of 95% or more (P<0.05) was considered significant.

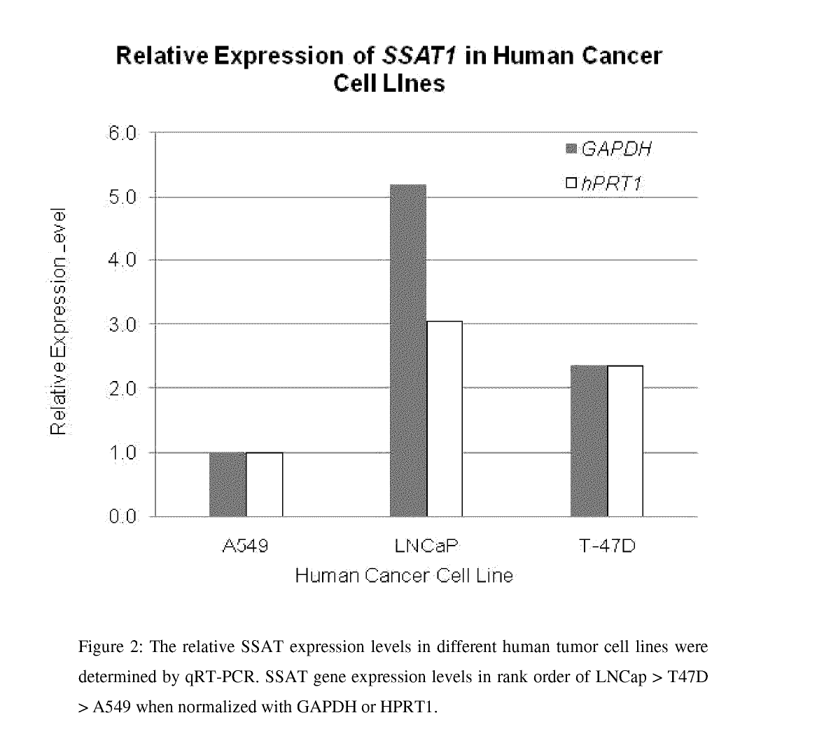

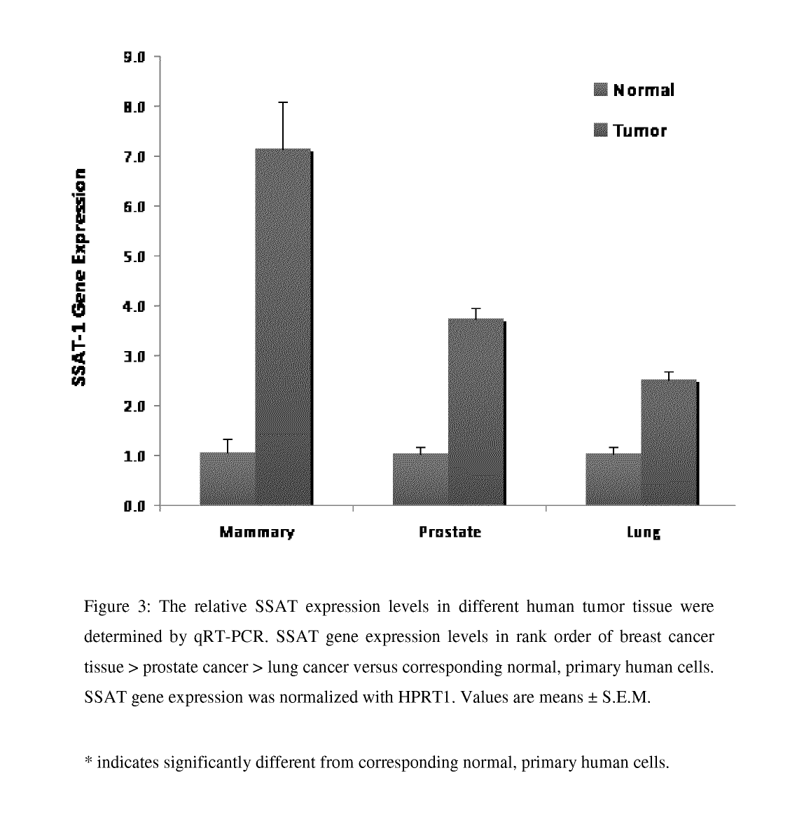

Referring to FIG. 2, high expression in the human cancer cell lines LNCaP (human prostate adenocarcinoma)>T47D (human breast epithelial cancer)>A549 (human lung epithelial cancer) was observed. Referring now to FIG. 3, with respect to patient-derived breast cancer tissue, lung cancer tissue, and prostate cancer tissue, an approximate seven-fold higher SSAT gene expression in breast cancer tissue versus normal, primary human mammary epithelial cells when normalized with hypoxanthine-guanine phosphoribosyltransferase (HPRT1) was observed. In addition, an approximate four fold and three fold higher SSAT gene expression was seen in prostate and lung cancer tissue versus normal, primary human prostate epithelial cells and normal, primary human bronchial/tracheal epithelial cells, respectively, when normalized with HPRT1. The particulars of patients and cancer tissue are set out in the table below.

| | GENDER | TYPE AND STAGE |
|---|---|---|
| AGE | (M/F) | OF CANCER |
| 56 | F | Breast Infil. Ductal (IV) |
| 38 | F | Breast Infil. Ductal (IIA) |
| 50 | F | Breast Infil. Ductal (I) |
| 75 | F | Lung Adenocarcinoma (IIB) |
| 46 | F | Lung Adenocarcinoma (IA) |
| 69 | F | Lung Adenocarcinoma (IIB) |
| 60 | M | Prostate Adenocarcinoma (IV) |
| 66 | M | Prostate Adenocarcinoma (IV) |
| 67 | M | Prostate Adenocarcinoma (IV) |

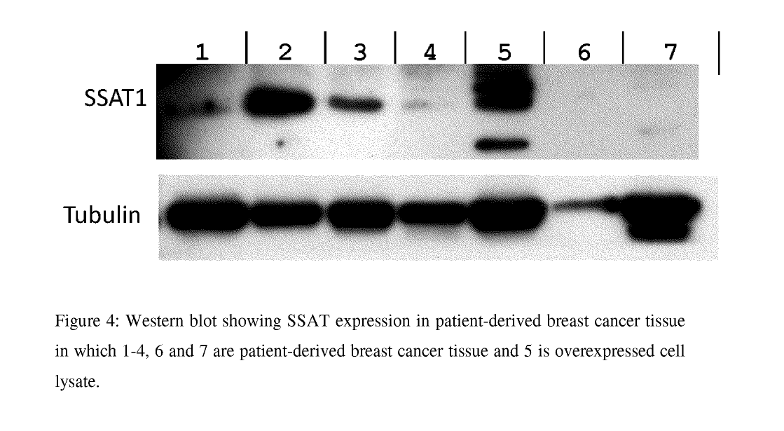

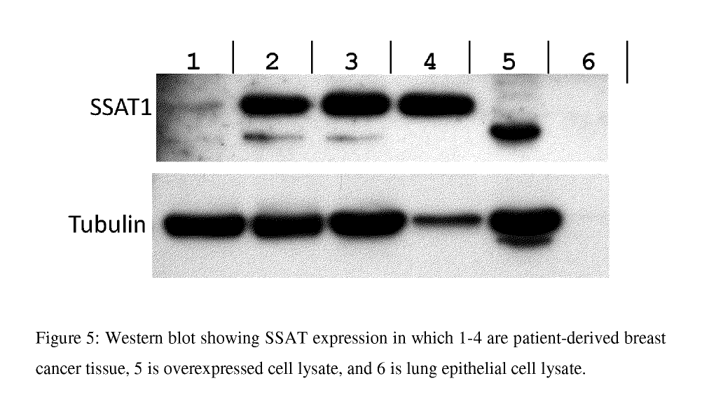

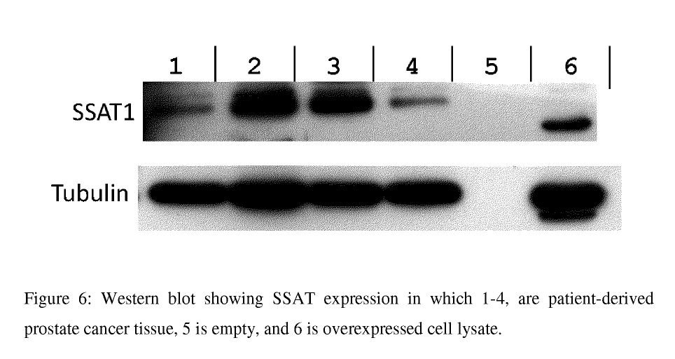

Western blots were also obtained for the patient-derived breast cancer tissue, lung cancer tissue, and prostate cancer tissue and are shown in FIGS. 4 to 6. Tubulin was used as reference gene for control purposes. The Western blot data shows elevated levels of SSAT protein in certain samples. This appears to correlate with the elevated mRNA levels. When normalized to the reference gene, tubulin, the gene expression data indicates that average SSAT gene expression is 4-fold higher in breast cancer cells relative to normal breast epithelial cells, almost 10-fold higher in prostate cancer cells relative to normal prostate cells, and approximately 4-fold higher in lung cancer cells relative to normal lung cells.

The quantified data of the Western blots expressed as a ratio of the internal control, tubulin, as the housekeeping protein is shown below.

| Cancer | Sample ID | SSAT1 | Tubulin | SSAT1/Tubulin |
|---|---|---|---|---|
| BREAST | 1 | 771.8567 | 1170.975 | 0.659157367 |
| | 2 | 1391.518 | 822.366 | 1.692091547 |
| | 3 | 245.4374 | 901.8199 | 0.272157873 |
| | 4 | 103.645 | 781.8483 | 0.132564146 |
| LUNG | 1 | 261.2871 | 760.1228 | 0.34374329 |
| | 2 | 926.3537 | 632.0238 | 1.465694297 |
| | 3 | 1152.342 | 807.9831 | 1.426195187 |
| | 4 | 1144.429 | 208.8031 | 5.480899899 |
| PROSTATE | 1 | 548.8936 | 791.1373 | 0.693803173 |
| | 2 | 1208.612 | 1145.081 | 1.055481249 |
| | 3 | 953.8715 | 941.5218 | 1.013116752 |
| | 4 | 126.2858 | 825.8522 | 0.152915726 |

The increased levels of SSAT mRNA expression in patient-derived tumors provide further evidence that elevated SSAT gene expression levels in cancer tissue may serve as a diagnostic marker for the detection of cancer and that SSAT may be a target for anti-cancer drug development.

It will be understood by a person skilled in the art that many of the details provided above are by way of example only, and are not intended to limit the scope of the invention which is to be determined with reference to the following claims.

What is claimed is:

1. A method of detecting cancer comprising correlating spermidine/spermine N¹-acetyltransferase gene expression to cancer.

2. The method as claimed in claim 1 including correlating elevated levels of spermidine/spermine N¹-acetyltransferase gene expression to cancer.

3. The method as claimed in claim 1 including correlating spermidine/spermine N¹-acetyltransferase gene expression to breast cancer.

4. The method as claimed in claim 1 including correlating spermidine/spermine N¹-acetyltransferase gene expression to lung cancer.

5. The method as claimed in claim 1 including correlating spermidine/spermine N¹-acetyltransferase gene expression to prostate cancer.
